(19) United States
(12) Patent Application Publication
(10) Pub. No.: US 2019/0216977 A1
(43) Pub. Date: Jul. 18, 2019

(54) NERVE TREATMENT DEVICES AND METHODS

(71) Applicant: LifeCell Corporation, Madison, NJ (US)

(72) Inventors: Hui Xu, Plainsboro, NJ (US); Wendell Sun, Warrington, PA (US); Cunqi Cui, Plainsboro, NJ (US); Hua Wan, Princeton, NJ (US)

(21) Appl. No.: 16/256,147

(22) Filed: Jan. 24, 2019

Related U.S. Application Data
(63) Continuation of application No. 12/956,058, filed on Nov. 30, 2010.
(60) Provisional application No. 61/266,348, filed on Dec. 3, 2009.

(51) Int. Cl. A61L 27/36; A61L 31/00; A61L 27/38

(52) U.S. Cl. CPC A61L 27/3675; A61L 31/005; A61L 2430/40; A61L 27/3625; A61L 2430/32; A61L 27/3834

(57) ABSTRACT

Devices and methods for treating defects in peripheral nerves are provided. The devices can include acellular arterial tissue matrices that facilitate regrowth of nerve tissue across a gap or defect in a peripheral nerve.

This application is a continuation of U.S. patent application Ser. No. 12/956,058, which was filed on Nov. 30, 2010, which claims priority under 35 U.S.C. § 119 to U.S. Provisional Patent Application No. 61/266,348, which was filed on Dec. 3, 2009.

Gaps or defects in peripheral nerves, due, for example, to trauma or surgery, are often treated using autologous nerve grafts. However, autografts require sacrifice of a healthy nerve with resultant permanent functional impairment. In addition, when multiple nerves are involved, it may not be possible to obtain a sufficient number of autografts. Further, synthetic or processed materials, which may be used as conduits for nerve regeneration, have had limited success in treating relatively long gap defects.

The present disclosure provides improved devices and methods for treating defects in peripheral nerves.

In certain embodiments, a method for treating a nerve is provided. The method comprises selecting a peripheral nerve having a gap across a portion of its length; and positioning an arterial tissue matrix across the gap, wherein substantially all of the native cells have been removed from the tissue matrix.

In certain embodiments a device for treating a nerve is provided, comprising an arterial tissue matrix, wherein substantially all of the native cells have been removed.

### DESCRIPTION OF THE DRAWINGS

FIG. 1 shows paw prints of rats after treatment with various graft materials, as described in Example 1.

FIGS. 2A and 2B are thigh circumference measurements for rats treated with various graft materials, as described in Example 1.

FIGS. 2C and 2D are lower leg circumference measurements for rats treated with various graft materials, as described in Example 1.

FIG. 3 is a bar graph showing percent gastrocnemius muscle recovery for rats treated with various graft materials, as described in Example 1.

FIG. 4A is a hematoxylin and eosin stained tissue section of a rat sciatic nerve defect without subsequent treatment, as described in Example 1.

FIG. 4B is a hematoxylin and eosin stained tissue section of a rat sciatic nerve after treatment with a nerve autograft, as described in Example 1.

FIG. 4C is a hematoxylin and eosin stained tissue section of a rat sciatic nerve defect after treatment with an acellular porcine nerve conduit, as described in Example 1.

FIG. 4D is a hematoxylin and eosin stained tissue section of a rat sciatic nerve defect after treatment with an acellular porcine artery conduit, as described in Example 1.

FIG. 4E is a hematoxylin and eosin stained tissue section of a rat sciatic nerve defect after treatment with an acellular porcine artery conduit filled with porcine acellular dermis paste, as described in Example 1.

FIG. 4F is a hematoxylin and eosin stained tissue section of a rat sciatic nerve defect after treatment with an acellular porcine dermal conduit, as described in Example

FIG. 5A is a hematoxylin and eosin stained tissue section of a rat sciatic nerve defect without subsequent treatment, as described in Example 1.

FIG. 5B is a hematoxylin and eosin stained tissue section of a rat sciatic nerve defect after treatment with a nerve autograft, as described in Example 1.

FIG. 5C is a hematoxylin and eosin stained tissue section of a rat sciatic nerve defect after treatment with an acellular porcine nerve, as described in Example 1.

FIG. 5D is a hematoxylin and eosin stained tissue section of a rat sciatic nerve defect after treatment with an acellular porcine artery conduit, as described in Example 1.

FIG. 5E is a hematoxylin and eosin stained tissue section of a rat sciatic nerve defect after treatment with an acellular porcine artery conduit filled with porcine acellular dermis paste, as described in Example 1.

FIG. 5F is a hematoxylin and eosin stained tissue section of a rat sciatic nerve defect after treatment with an acellular porcine dermal conduit, as described in Example 1.

FIG. 6A is a Bodian stained tissue section of a rat sciatic nerve defect without subsequent treatment, as described in Example 1.

FIG. 6B is a Bodian stained tissue section of a rat sciatic nerve defect after treatment with a nerve autograft, as described in Example 1.

FIG. 6C is a Bodian stained tissue section of a rat sciatic nerve defect after treatment with an acellular porcine nerve, as described in Example 1.

FIG. 6D is Bodian stained tissue section of a rat sciatic nerve defect after treatment with an acellular porcine artery conduit, as described in Example 1.

FIG. 6E is a Bodian stained tissue section of a rat sciatic nerve defect after treatment with an acellular porcine artery conduit filled with porcine acellular dermis paste, as described in Example 1.

FIG. 6F is a Bodian stained tissue section of a rat sciatic nerve defect after treatment with an acellular porcine dermal conduit, as described in Example 1.

FIG. 7A is a neurofilament stained (anti-NF200) tissue section of a rat sciatic nerve defect without subsequent treatment, as described in Example 1.

FIG. 7B is a neurofilament stained (anti-NF200) tissue section of a rat sciatic nerve defect after treatment with a nerve autograft, as described in Example 1.

FIG. 7C is a neurofilament stained (anti-NF200) tissue section of a rat sciatic nerve defect after treatment with an acellular porcine nerve, as described in Example 1.

FIG. 7D is a neurofilament stained (anti-NF200) tissue section of a rat sciatic nerve defect after treatment with an acellular porcine artery, as described in Example 1.

FIG. 7E is a neurofilament stained (anti-NF200) tissue section of a rat sciatic nerve defect after treatment with an acellular porcine artery filled with porcine acellular dermal matrix paste, as described in Example 1.

FIG. 7F is a neurofilament stained (anti-NF200) tissue section of a rat sciatic nerve defect after treatment with a porcine acellular dermal matrix, as described in Example 1.

FIG. 8A is a tissue section of a rat sciatic nerve defect stained with anti-GFAP antibodies after treatment with a nerve autograft, as described in Example 1.

FIG. 8B is a tissue section of a rat sciatic nerve produced defect with anti-GFAP antibodies after treatment with an acellular porcine artery, as described in Example 1.

### DESCRIPTION OF CERTAIN EXEMPLARY EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit, unless specifically stated otherwise. Also the use of the term “portion” may include part of a moiety or the entire moiety. Any range described herein will be understood to include the endpoints and all values between the endpoints.

The term “acellular tissue matrix,” as used herein, refers generally to any tissue matrix that is substantially free of native cells. Acellular tissue matrices may be derived from human or xenogenic sources. Acellular tissue matrices may be seeded with exogenous cells derived from the recipient or other sources.

In various embodiments, methods for repairing defects or gaps in peripheral nerves are provided. In some embodiments, the methods can include regeneration of a portion of one or more nerve fibers lost due to, but not limited to, trauma, surgery, or disease. In certain embodiments, the methods can include regeneration of nerve tissue to repair a gap or defect in a nerve fiber. In various embodiments, the methods allow at least partial restoration of function provided by a nerve, including sensory, somatosensory, and/or motor functions.

As used herein, the terms “gap” or “defect” in a nerve will be used interchangeably and will be understood to include any section of a peripheral nerve that has been rendered dysfunctional due to any type of damage to that section of nerve. The “gap” or “defect” may include a structural gap, in which part of the nerve is absent (due, for example, to the nerve being severed or dying due to any damaging process) or may include a functional gap or defect, wherein the nerve may be present but may not function properly. Further, the “gap” or “defect” can include a gap or defect between two segments of functional nerves or between a distal or proximal portion of a nerve and tissue affected by the nerve, e.g., between a terminal portion of a nerve and a muscle or other tissue.

In certain embodiments, the methods can include identifying a gap or defect in a nerve fiber and positioning an arterial tissue matrix across the region of the gap or defect to facilitate repair, regrowth, or regeneration of the nerve fiber. The arterial tissue matrix can include an acellular arterial tissue matrix, wherein substantially all of the native cells have been removed. In some embodiments, the arterial tissue matrix can form a tube or conduit through which a peripheral nerve can grow when the conduit is implanted across a defect in the peripheral nerve.

In various embodiments, the arterial tissue matrix can allow a peripheral nerve to grow or regenerate across a defect to produce a certain level of functional recovery. In various embodiments, the matrix can allow at least 50% functional recovery, at least 60% functional recovery, at least 70% functional recovery, or at least 80% functional recovery, or any ranges between these values. The functional recovery may be quantified in various ways. In some embodiments, functional recovery is quantified using the size or strength of a muscle innervated by a treated nerve. In certain embodiments, functional recovery is measured by comparing the dry weight or volume of a muscle innervated by the nerve after recovery with the dry weight of a corresponding muscle either before a defect occurred or on an opposing, unaffected limb. In other embodiments, functional recovery is assessed by detection of pain sensation.

The arterial tissue matrix can be produced by treating a section of an artery to remove substantially all of the cells and certain other antigenic materials to produce an acellular arterial tissue matrix. The section of artery can be selected from a variety of different anatomic sites, and can be derived from human and/or non-human sources, as described further below. In certain embodiments, the section of artery is selected based on a desired size (e.g., length of defect to be treated and/or approximate tubular diameter of nerve or nerves to be treated). In various embodiments, the gap or defect can be greater than 1 cm, greater than 2 cm, between 0.1 cm and 1 cm, between 1 cm and 2 cm, greater than 4 cm, greater than 6 cm, greater 10 cm, or any ranges in between. Suitable arterial sites can include, but are not limited to, carotid, femoral, ulnar, median, and/or brachial arteries.

To treat a gap or defect in a nerve, the nerve to be treated may first be cleaned to remove damaged tissue and/or excise existing portions of defective nerve, if present. Next, an acellular arterial tissue matrix can be placed within the gap or defect site to form a conduit across the gap or defect to allow nerve repair, regrowth, or regeneration through the arterial tissue matrix. Since arteries have a naturally tubular shape, in some embodiments, the acellular arterial tissue matrix is produced by procuring a tubular arterial section and treating the section to remove cells or other components without disrupting the tubular structure of the arterial tissue matrix. In various embodiments, the acellular arterial tissue matrix can be held in place using sutures or biocompatible adhesives (e.g., fibrin glue).

Various types of tissue conduits have been used to treat gaps or defects in peripheral nerves. However, in many cases, it was necessary to fill the tissue conduits with exogenous materials, such as hydrogels or other materials that are believed to support nerve regeneration. In certain embodiments, acellular arterial tissue matrices can provide suitable conduits for treatment of peripheral nerves without the need for additional materials to be placed within the conduits. In certain embodiments, the acellular arterial tissue matrices can be filled with particulate and/or pastes formed from acellular tissue matrices.

In certain embodiments, gaps or defects in nerves can be treated without supplying additional cells (e.g., stem cells) to the acellular arterial tissue matrix. In some embodiments, the acellular arterial tissue matrices can be seeded with certain cells that facilitate nerve repair, regrowth, or regeneration. In certain embodiments, the acellular arterial tissue matrices can be seeded with stem cells, such as mesenchymal stems cells such as, for example, embryonic stem cells, adult stem cells isolated from bone marrow, fat or other tissue, and neuronal cells. In various embodiments, autologous stems cells may be used. In some embodiments, allogenic cells can be pre-seeded to the grafts and cultured in vitro and lysed before implantation. Growth factors promoting nerve regeneration can also be applied to the grafts with or without the cells. In various embodiments, the cells can be contained in biocompatible carriers such as bioglues, hydrogels, or extracellular matrix pastes, and the carriers can be placed within the grafts before or after implantation.

In various embodiments, the arterial tissue matrix seeded with certain cells can allow a peripheral nerve to grow or regenerate across a defect to produce a certain level of functional recovery. In various embodiments, the matrix and cells can allow at least 50% functional recovery, at least 60% functional recovery, at least 70% functional recovery, or at least 80% functional recovery, or any ranges between these values. The functional recovery may be quantified in various ways. In some embodiments, functional recovery is quantified using the size or strength of a muscle innervated by a treated nerve. In certain embodiments, functional recovery is measured by comparing the dry weight or volume of a muscle innervated by the nerve after recovery with the dry weight of a corresponding muscle either before a defect occurred or on an opposing, unaffected limb. In other embodiments, functional recovery is assessed by detection of pain sensation.

While an acellular tissue matrix may be made from one or more individuals of the same species as the recipient of the acellular tissue matrix graft, this is not necessarily the case. Thus, for example, an acellular tissue matrix may be made from porcine tissue and implanted in a human patient. Species that can serve as recipients of acellular tissue matrix and donors of tissues or organs for the production of the acellular tissue matrix include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice.

Arterial acellular tissue matrices suitable for use in the present disclosure can be produced by a variety of methods. In various embodiments, the arterial acellular tissue matrices can include various proteins other than collagen, which may support nerve regeneration. In some embodiments, the matrices can include glycosamionglycans (GAGs) and/or elastins, which are present in intact arterial tissue and/or include an intact basement membrane.

In general, the steps involved in the production of an acellular tissue matrix include harvesting the tissue from a donor (e.g., a human cadaver or animal source) and cell removal under conditions that preserve biological and structural function. In certain embodiments, the process includes chemical treatment to stabilize the tissue and avoid biochemical and structural degradation together with or before cell removal. In various embodiments, the stabilizing solution arrests and prevents osmotic, hypoxic, autolytic, and proteolytic degradation, protects against microbial contamination, and reduces mechanical damage that can occur with tissues that contain, for example, smooth muscle components (e.g., blood vessels). The stabilizing solution may contain an appropriate buffer, one or more antioxidants, one or more oncotic agents, one or more antibiotics, one or more protease inhibitors, and/or one or more a smooth muscle relaxant.

The tissue is then placed in a decellularization solution to remove viable cells (e.g., epithelial cells, endothelial cells, smooth muscle cells, and fibroblasts) from the structural matrix without damaging the biological and structural integrity of the collagen matrix. The decellularization solution may contain an appropriate buffer, salt, an antibiotic, one or more detergents (e.g., TRITONX-100™, sodium deoxycholate, polyoxyethylene (20) sorbitan mono-oleate), one or more agents to prevent cross-linking, one or more protease inhibitors, and/or one or more enzymes. In some embodiments, the decellularization solution comprises 1% TRITON X-100™ in RPMI media with Gentamicin and 25 mM EDTA (ethylenediaminetetraacetic acid). In some embodiments, the tissue is incubated in the decellularization solution overnight at 37° C. with gentle shaking at 90 rpm. In certain embodiments, additional detergents may be used to remove fat from the tissue sample. For example, in some embodiments, 2% sodium deoxycholate is added to the decellularization solution.

After the decellularization process, the tissue sample is washed thoroughly with saline. In some exemplary embodiments, e.g., when xenogenic material is used, the decellularized tissue is then treated overnight at room temperature with a deoxyribonuclease (DNase) solution. In some embodiments, the tissue sample is treated with a DNase solution prepared in DNase buffer (20 mM HEPES (4-(2-hydroxyethyl)-1-piperazineethanesulfonic acid), 20 mM CaCl₂) and 20 mM MgCl2). Optionally, an antibiotic solution (e.g., Gentamicin) may be added to the DNase solution. Any suitable buffer can be used as long as the buffer provides suitable DNase activity.

Elimination of the α-gal epitopes from the collagen-containing material may diminish the immune response against the collagen-containing material. The α-gal epitope is expressed in non-primate mammals and in New World monkeys (monkeys of South America) as well as on macromolecules such as proteoglycans of the extracellular components. U. Galili et al., J. Biol. Chem. 263: 17755 (1988). This epitope is absent in Old World primates (monkeys of Asia and Africa and apes) and humans, however. Id. Anti-gal antibodies are produced in humans and primates as a result of an immune response to α-gal epitope carbohydrate structures on gastrointestinal bacteria. U. Galili et al., Infect. Immun. 56: 1730 (1988); R. M. Hamadeh et al., J. Clin. Invest. 89: 1223 (1992).

Since non-primate mammals (e.g., pigs) produce α-gal epitopes, xenotransplantation of collagen-containing material from these mammals into primates often results in rejection because of primate anti-Gal binding to these epitopes on the collagen-containing material. The binding results in the destruction of the collagen-containing material by complement fixation and by antibody dependent cell cytotoxicity. U. Galili et al., Immunology Today 14: 480 (1993); M. Sandrin et al., Proc. Natl. Acad. Sci. USA 90: 11391 (1993); H. Good et al., Transplant. Proc. 24: 559 (1992); B. H. Collins et al., J. Immunol. 154: 5500 (1995). Furthermore, xenotransplantation results in major activation of the immune system to produce increased amounts of high affinity anti-gal antibodies. Accordingly, in some embodiments, when animals that produce α-gal epitopes are used as the tissue source, the substantial elimination of α-gal epitopes from cells and from extracellular components of the collagen-containing material, and the prevention of re-expression of cellular α-gal epitopes can diminish the immune response against the collagen-containing material associated with anti-gal antibody binding to α-gal epitopes.

To remove α-gal epitopes, after washing the tissue thoroughly with saline to remove the DNase solution, the tissue sample may be subjected to one or more enzymatic treatments to remove certain immunogenic antigens, if present in the sample. In some embodiments, the tissue sample may be treated with an α-galactosidase enzyme to eliminate α-gal epitopes if present in the tissue. In some embodiments, the tissue sample is treated with α-galactosidase at a concentration of 300 U/L prepared in 100 mM phosphate buffer at pH 6.0 In other embodiments, the concentration of α-galactosidase is increased to 400 U/L for adequate removal of the α-gal epitopes from the harvested tissue. Any suitable enzyme concentration and buffer can be used as long as sufficient removal of antigens is achieved.

Alternatively, rather than treating the tissue with enzymes, animals that have been genetically modified to lack one or more antigenic epitopes may be selected as the tissue source. For example, animals (e.g., pigs) that have been genetically engineered to lack the terminal α-galactose moiety can be selected as the tissue source. For descriptions of appropriate animals see co-pending U.S. application Ser. No. 10/896,594 and U.S. Pat. No. 6,166,288, the disclosures of which are incorporated herein by reference in their entirety.

After the acellular tissue matrix is formed, histocompatible, viable cells may optionally be seeded in the acellular tissue matrix to produce a graft that may be further remodeled by the host. In some embodiments, histocompatible viable cells may be added to the matrices by standard in vitro cell co-culturing techniques prior to transplantation, or by in vivo repopulation following transplantation. In vivo repopulation can be by the recipient's own cells migrating into the acellular tissue matrix or by infusing or injecting cells obtained from the recipient or histocompatible cells from another donor into the acellular tissue matrix in situ. Various cell types can be used, including embryonic stem cells, adult stem cells (e.g. mesenchymal stem cells), and/or neuronal cells. In various embodiments, the cells can be directly applied to the inner portion of the acellular tissue matrix just before or after implantation. In certain embodiments, the cells can be placed within the acellular tissue matrix to be implanted, and cultured prior to implantation.

The following examples are provided to better explain the various embodiments and should not be interpreted in any way to limit the scope of the present disclosure.

### Example 1: Use of Various Graft Materials for Repair of Peripheral Nerve Defects

To study the effectiveness of various graft materials to repair peripheral nerve defects in adult male Lewis rats animals were treated using either (1) rat sciatic nerve autograft (Auto) (2) porcine acellular nerves (APN), (3) porcine acellular artery (VC), (4) porcine acellular artery filled with porcine acellular dermal paste (VCP), (5) porcine acellular dermis with the epithelial basement membrane intact and sutured such that the basement membrane faced the outside of the conduit (PADM), (6) human acellular dermis with the epithelial basement membrane intact and sutured such that the basement membrane faced the inside of the conduit (HADM-in), (7) human acellular dermis with the epithelial basement membrane intact and sutured such that the basement membrane faced the outside of the conduit (HADM-out), or (8) porcine acellular artery seeded with rat mesenchymal stem cells isolated from animals from the same inbred strain (VCM). Untreated controls (Def) were also produced.

### Production of Acellular Graft Materials

(1) Acellular Artery and Nerve:

Porcine arteries and nerves were processed using the same protocol to produce either acellular artery or acellular nerve. Portions of pig carotid artery and were harvested from the distal end of pig carotid artery to match the size of the rat sciatic nerve to be treated. The vessels or nerves were soaked in 0.5× Vitrosol (citric acid 2.4 mM (0.5 g/L), sodium citrate 7.6 mM (2.24 g/L), EDTA 1 mM (2 ml of 0.5M EDTA), NaCl 100 mM (5.844 g/L), Tween 20 0.02% (180 ul/L), glycerin 35% (w/v) (280 ml/L), ethylene glycol 25% (w/v) (225 ml/L), PD-30 30% (300 g/L)) for about (2 hours) Samples were then equilibrated by shaking in 0.5× Vitrisol for 1-2 hours at 90 rpm. The Vitrosol was replaced with fresh 0.5× Vitrosol and shaken for an additional 1-2 hours. The 0.5× Vitrosol was replaced with 1× Vitrosol and shaken overnight. The Vitrisol was again replaced with fresh 1× Vitrosol (200 ml) and shaken for 2 hours, and was then stored at −80° C. overnight.

The vessels or nerves were thawed in a 37° C. water bath and washed three times with normal saline for 30 minutes each wash. NaCl was used for all washes to prevent precipitation of calcium phosphate upon implantation, which may occur if PBS were used. Saline was aspirated from the vessels, and any remaining loose connective tissue was removed. Samples were placed in a decellularizing solution (1% TX-100 in RPMI (Gentamicin) with 25 mM EDTA) and incubated overnight at room temperature while shaking at 90 rpm. The decellularizing solution was aspirated, and samples were washed again to remove detergents. The wash was performed for at least three hours with six changes of saline.

Vessels or nerves were treated with DNase (30 U/ml) (Genentech, CA) in DNase buffer (20 mM HEPES, 20 mM CaCl₂), 20 mM MgCl2, pH 7.5) overnight at 37° C. with gentle shaking (90 rpm). Gentamicin was added to a final concentration of 50 μg/ml. The DNase solution was aspirated and samples were washed with an equal volume of saline three times for 30 minutes each wash. Vessels or nerves were then treated with α-galactosidase (200 U/L) in a 100 mM phosphate buffer (pH 6) overnight at 37° C. with gentle shaking (90 rpm). The α-galactosidase solution was aspirated, and the vessels or nerves were washed with an equal volume of saline three times for 30 minutes each wash.

Vessels or nerves were then rinsed with a storage solution (citric acid 7.2 mM (1.51 g/L), sodium citrate 22.8 mM (6.71 g/L), EDTA 3 mM (1.12 g/L), NaCl 50 mM (8.77 g/L), Tween-20 0.03% (w/v) (276 ul/L), glycerol 15% (w/v) (120 ml/L), trehalose 750 mM (283.75 g/L), pH5.4). Samples were then equilibrated in the storage solution for 2 hr. The storage solution was then replaced with fresh solution, and samples were equilibrated overnight at room temperature. Finally, samples were placed in fresh storage solution and stored at room temperature or 4° C.

(2) Production of Acellular Dermal Materials:

Porcine or human skin was used for production of acellular dermal materials. For human dermal matrices, Alloderm®, a human acellular tissue matrix produced by LifeCell Corporation (Branchburg, N.J.) was used. For porcine tissues, the epithelial cells were removed by soaking overnight in 1 M NaCl solution at room temperature, and the basement membrane was left intact.

The porcine samples were then placed in a decellularization solution (2% sodium deoxylate in HEPES with Gentamicin and 25 mM EDTA (ethylenediaminetetraacetic acid)) overnight at 37° C. with gentle shaking at 90 rpm. After the decellularization process, the tissue samples were washed thoroughly with saline, and porcine skin was treated with DNase and α-galactosidase, as described above for arteries and nerves.

(3) Production of Acellular Artery Filled with PADM Paste:

Acellular arterial tissue matrices were produced as described above in section (1). Porcine acellular dermis, produced as described in section (2) above, was then micronized by freeze-drying and pulverizing using a cryomill. The pulverized materials was suspended in sterile saline.

(4) Seeding with Mesenchymal Stem Cells:

Porcine acellular arterial matrices were produced as described above in section (1). The matrices were seeded with rat mesenchymal stem cells (MSCs) obtained from rats from the same inbred strain as those in which the nerve defects were produced. To seed the matrices, cultured rat MSCs were trypsinized and resuspended in Mesenchymal stem cell expansion medium (Millipore, Mass.) at a density of 5×10⁶ cells/ml. One end of the graft was sutured to one end of the nerve in which a defect was created defect, and one hundred microliters of cells placed within the conduit. After the cells were placed within the graft, the other (open) end of the vessel was sutured to the other end of the nerve.

A sciatic nerve defect was created in each rat by cutting and removing 1.0-1.2 cm of nerve. The left sciatic nerve was damaged and treated in each animal, and the right sciatic nerve of the same animal was left intact for comparison. All animals were adult male Lewis rats. The proximal and distal axons were sutured together by a 12 to 15 mm long porcine vessel or nerve graft with end-to-end anastomoses using 9-0 nylon interrupted sutures (Micruns, Chicago, Ill.). In the autograft group, the rat sciatic nerve was reconnected following the creation of the nerve defect.

Thirty-five rats were used in total and were treated or left as untreated controls, as outlined in Table 1.

TABLE 1
Outline of treatment groups

| | |
|---|---|
| 1. | Autografts; n = 3 (Auto) |
| 2. | Acellular porcine nerve; n = 5 (APN) |
| 3. | Acellular porcine artery; n = 5 (VC) |
| 4. | Acellular porcine artery + 10% of PADM paste by weight; |
| | n = 5 (VCP) |
| 5. | Sutured PADM; n = 3 (PADM) |
| 6. | Sutured HADM, basement membrane inside (n = 3) |
| 7. | Sutured HADM, basement membrane outside (n = 3) |
| 8. | Acellular porcine artery + rat stem cells (n = 5) (VCM) |
| 9. | No repair (control); n = 3 (Def) |

### Assessment of Nerve Regeneration:

Various functional and structural measurements were performed on the animals, as described below. At 4 months, the animals were sacrificed for histologic evaluation. Certain functional parameters assessed during the treatment period are described in Table 2, below. These parameters included painful refection, autotomy, presence of foot ulcers, strephexopodia, walking ability, resistant strain, and ability to turn over. Painful refection was assessed by a pain sensitivity test that evaluated the response of animals to a needle poke on the sole of the foot; the time at which withdrawal from the poke returned indicated recovery of sensory function. Resistant strain was assessed by examining and comparing the leg strength of the control leg and experimental leg. The turn-over test was performed by lying the rat back down to see if the animals could turn over normally. Other tests were observations by a surgeon.

TABLE 2
Functional Parameter Assessment

| | Painful | | Feet | | Walking | Resistant | Turn |
|---|---|---|---|---|---|---|---|
| Group | refection | Autotomy | ulcer | Strephexopodia | (limp) | strain | over |
| Def | No | 2/3 | 2/3 | No | No | weak | Normal |
| | | | | recovery | recovery | | |
| Auto | 12 weeks | 0/3 | 0/3 | 14 | 14 | 16 weeks | Normal |
| | | | | weeks | weeks | | |
| APN | 16 weeks | 0/5 | 0/5 | 16 | 16 | 16 weeks | Normal |
| | | | | weeks | weeks | | |
| VC | 16 weeks | 0/5 | 0/5 | 16 | 16 | 16 weeks | Normal |
| | | | | weeks | weeks | | |
| VCP | No | 0/3 | 0/3 | 16 | 16 | 16 weeks | Normal |
| | | | | weeks | weeks | | |
| PADM | No | 1/3 | 1/3 | No | No | weak | Normal |
| | | | | recovery | recovery | | |
| HADM- | 16 weeks | 0/3 | 0/3 | 16 | 16 | 16 weeks | Normal |
| in | | | | weeks | weeks | | |
| HADM- | No | 2/3 | 2/3 | No | No | weak | Normal |
| out | | | | recovery | recovery | | |
| VC- | 12 weeks | 0/5 | 0/5 | 14 | 14 | 16 weeks | Normal |
| SMCs | | | | weeks | weeks | | |

Rats implanted with autografts showed pain reflection by 12 weeks, while rats implanted with acellular porcine nerve, acellular porcine artery, or human acellular dermis with basement membrane on the inside of the conduit showed pain reflection by 16 weeks with no sign of autotomy and feet ulcer. The rats treated with acellular porcine artery plus of stem cells had equivalent recovery rates to the rats treated with autografts. Rats implanted with sutured dermal tissue with basement membrane facing out showed no pain reflection by the date of sacrifice and had high incidences of autotomy. In addition, like the defect group, foot ulcers were seen in animals treated with sutured dermal tissue with the basement membrane facing out. Although animals treated with acellular porcine artery filled with dermal paste did not show pain reflection, no autotomy or foot ulcers were seen in these animals. Rats that were left untreated or were treated with porcine acellular dermis developed foot ulcers.

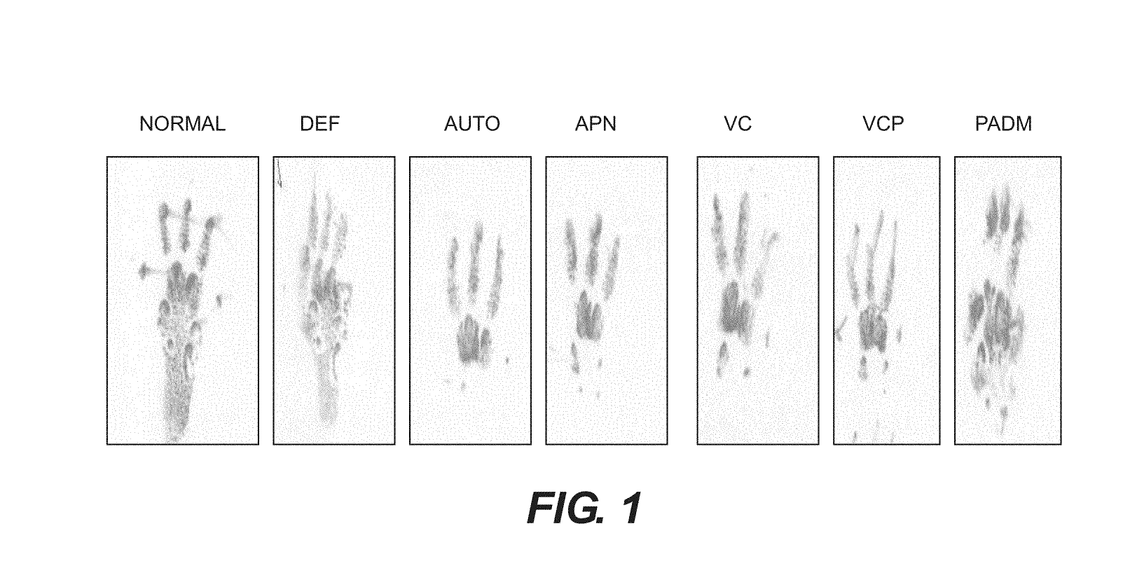

Paw prints of animals at day 0, 7, 14, 28 and 42 were produced to assess sciatic nerve function. FIG. 1 shows paw prints of rats at 42 days after treatment with various graft materials. The similarity between treated rats and normal rat foot prints indicates a degree of sciatic nerve functional recovery. As shown, rats treated with an autograft, which is the current gold-standard treatment for peripheral nerve defects, and acellular porcine artery had similar paw prints, indicating similar functional recovery in these groups compared to other treatment groups.

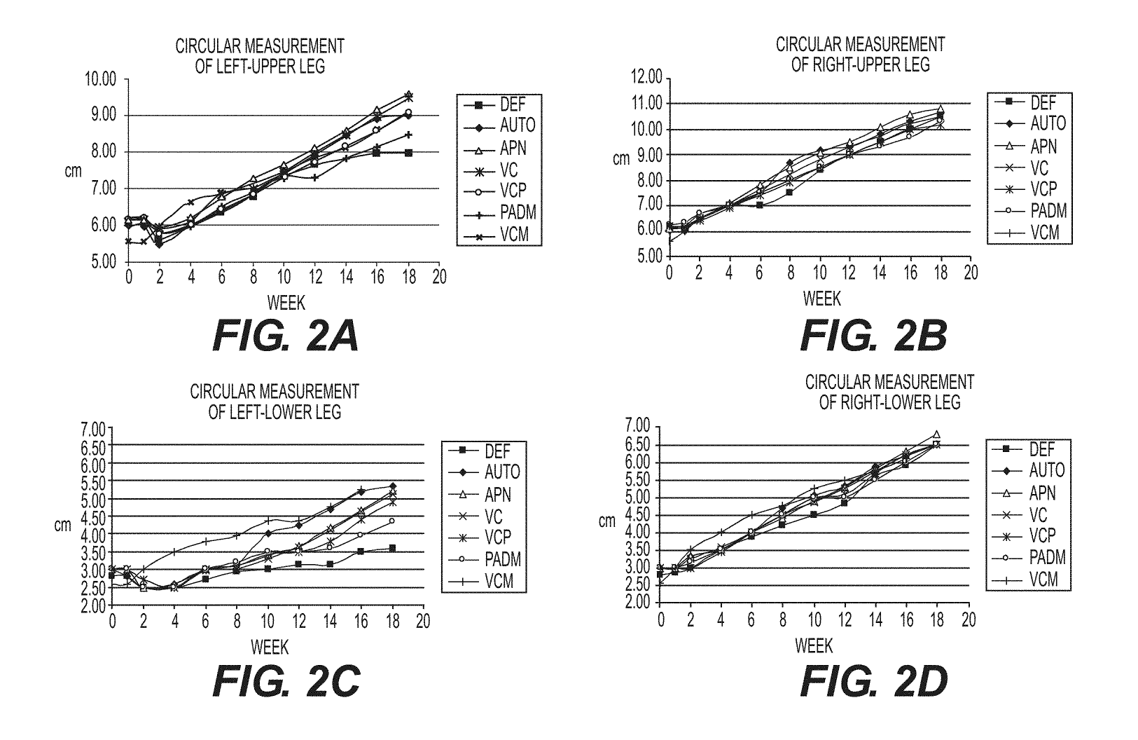

Limb circumference, measured at the lower limb or thigh, was used to assess functional recovery. Greater limb circumference was considered indicative of muscle growth, and therefore, greater nerve regeneration. FIGS. 2A and 2B are thigh circumference measurements for rats treated with various graft materials. FIG. 2A provides measurements for the left thigh in which the sciatic nerve was disrupted, as described above, and FIG. 2B provides measurements for the right thigh, in which the sciatic nerve was intact. FIGS. 2C and 2D are lower leg circumference measurements for rats treated with various graft materials. FIG. 2C provides measurements for the left limb in which the sciatic nerve was disrupted, as described above, and FIG. 2D provides measurements for the right limb, in which the sciatic nerve was intact. As shown, animals treated with acellular porcine artery had an increase in thigh and lower limb circumference measurements in the treated limb that was similar to that seen in animals treated with autografts.

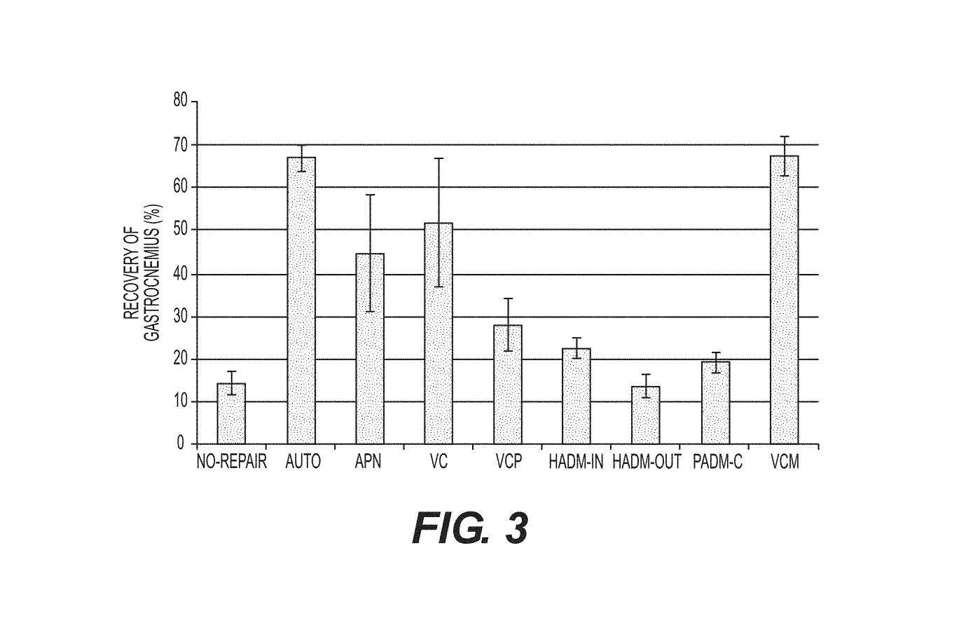

FIG. 3 is a bar graph showing percent gastrocnemius muscle recovery for rats treated with various graft materials, as described in Example 1. The percent muscle recovery was measured by determining the ratio of the dry weight of the treated (left) limb gastrocnemius muscle to the dry weight of the control (right) limb gastrocnemius muscle. All treatment groups except the group treated with HADM-Out demonstrated functional recovery as compared to the control (untreated) group. Autografts provided close to 70% recovery. Acellular porcine artery (VC) showed similar recovery of gastrocnemius weight as autografts. When dermal tissue with the basement membrane facing inside was used, slight (22%) functional recovery was observed, but when dermal tissue with the basement membrane facing outside was used, no nerve function recovery was observed. In animals treated with acellular porcine artery seeded with stem cell, the functional recovery was equal to that observed with autografts. In addition, the recovery of gastrocnemius weight for animals treated with acellular porcine artery was surpassed only by that of the autograft group and the group treated with acellular artery plus of stem cells.

TABLE 3
Weight of Gastrocnemius in Treatment Groups

| | Average | StDev | N |
|---|---|---|---|
| No-repair | 14.2 | 2.8 | 3 |
| Auto | 66.9 | 3 | 3 |
| APN | 44.6 | 13.6 | 5 |
| VC | 51.7 | 14.9 | 4 |
| VCP | 27.9 | 6.1 | 5 |
| PADM-C | 19.1 | 2.3 | 3 |
| VCM | 67.2 | 4.6 | 4 |

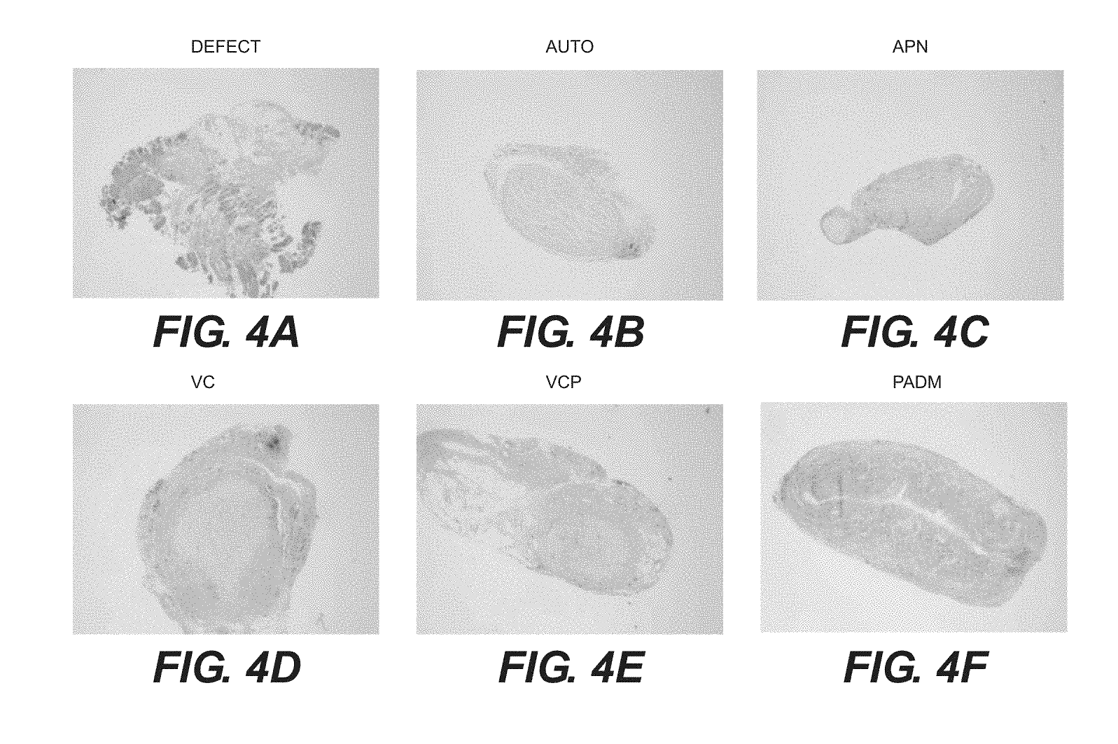

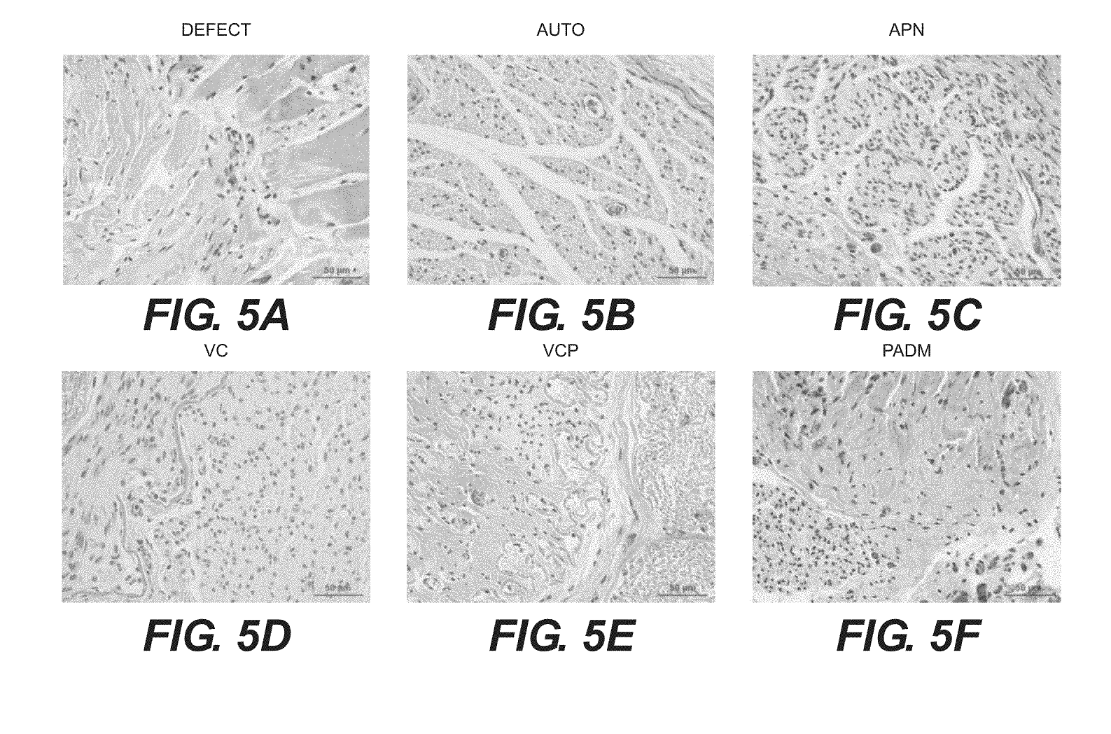

Various histological analyses were performed on the sacrificed animals. All sections shown in FIGS. 4A-5F were hematoxylin and eosin stained. FIG. 4A is a tissue section of a rat sciatic nerve defect without subsequent treatment, and FIG. 5A is a tissue section of a rat sciatic nerve defect without subsequent treatment at a higher magnification. FIG. 4B is a tissue section of a rat sciatic nerve defect after treatment with a nerve autograft, and FIG. 5B is a tissue section of a rat sciatic nerve defect after treatment with a nerve autograft at a higher magnification. FIG. 4C is a tissue section of a rat sciatic nerve defect after treatment with an acellular porcine nerve conduit, and FIG. 5C is a tissue section of a rat sciatic nerve defect after treatment with an acellular porcine nerve conduit at a higher magnification. FIG. 4D is a tissue section of a rat sciatic nerve defect after treatment with an acellular porcine artery conduit, and FIG. 5D is a tissue section of a rat sciatic nerve defect after treatment with an acellular porcine artery conduit at a higher magnification. FIG. 4E is a tissue section of a rat sciatic nerve defect after treatment with an acellular porcine artery conduit filled with porcine acellular dermis paste, and FIG. 5E is a tissue section of a rat sciatic nerve defect after treatment with an acellular porcine artery conduit filled with porcine acellular dermis paste. FIG. 4F is a tissue section of a rat sciatic nerve defect after treatment with an acellular porcine dermal conduit, and FIG. 4F is a tissue section of a rat sciatic nerve defect after treatment with an acellular porcine dermal conduit at a higher magnification.

At four months post implantation, all treatment sites were filled with nerve fibers except for those treated with porcine acellular dermis with basement membrane on the outside (FIGS. 4F and 5F). In addition, arterial tissue matrices were filled with neurofibrils, similar to the pattern seen in nerve defects treated with autografts, indicating the nerve defect was completely bridged (FIGS. 5A-5F).

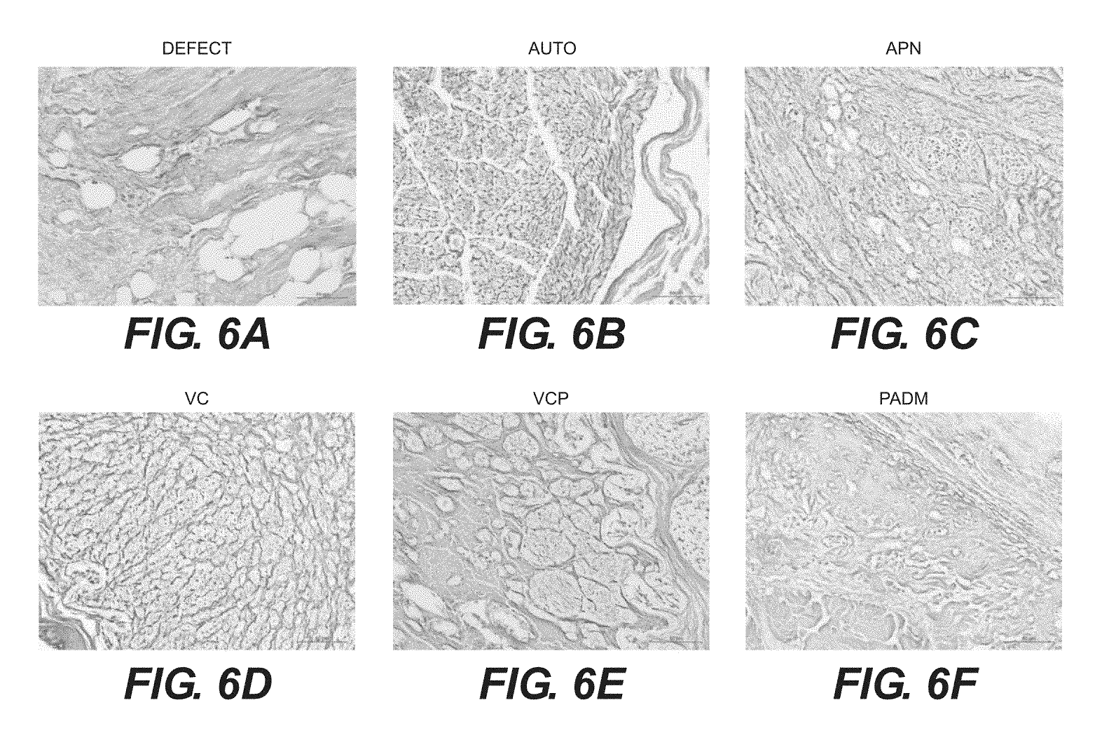

FIG. 6A-6F are Bodian stained tissue sections. FIG. 6A is a Bodian stained tissue section of a rat sciatic nerve defect without subsequent treatment. FIG. 6B is a Bodian stained tissue section of a rat sciatic nerve defect after treatment with a nerve autograft. FIG. 6C is a Bodian stained tissue section of a rat sciatic nerve defect after treatment with an acellular porcine nerve conduit. FIG. 6D is a Bodian stained tissue section of a rat sciatic nerve defect after treatment with an acellular porcine artery conduit. FIG. 6E is a Bodian stained tissue section of a rat sciatic nerve defect after treatment with an acellular porcine artery conduit filled with porcine acellular dermis paste. FIG. 6F is a Bodian stained tissue section of a rat sciatic nerve defect after treatment with an acellular porcine dermal conduit.

Normal nerve cross sections have a well organized nerve structure, but, when the nerve was cut without subsequent treatment, the regenerating nerve grew randomly into adjacent muscle (6A). The acellular porcine nerve matrix (6C) showed similar histologic structure as the nerve autograft (6B), although it is not clear the nerve fibers were the pre-implant porcine nerve or regenerated rat nerve. Bodian staining confirmed that tissue within the acellular porcine artery (6D) included neurofilaments, while dermal tissue paste did not promote additional nerve regeneration (6E). These results are consistent with the functional recovery of rat legs, indicating acellular porcine arteries can be used to support or guide nerve defect. Dermal material (6F) with the basement membrane facing outward did not appear to support nerve regrowth.

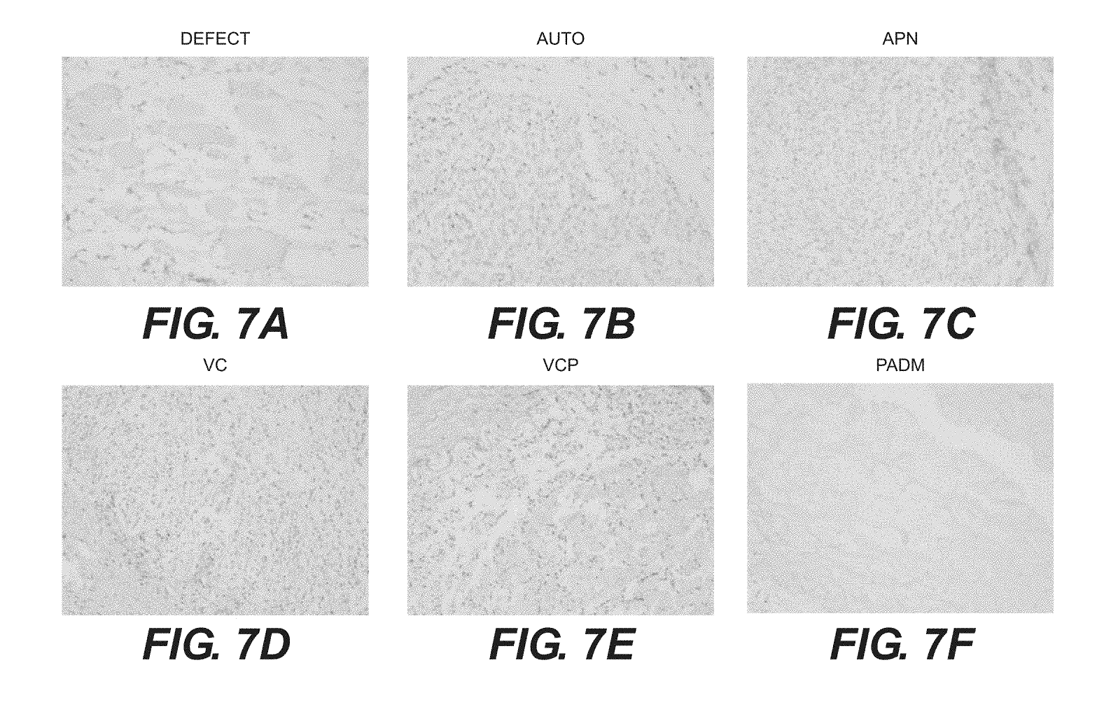

FIGS. 7A-7F are tissue sections stained with anti-NF200 antibodies to identified neurofilaments. FIG. 7A is a neurofilament stained (anti-NF200) tissue section of a rat sciatic nerve defect, produced as described in Example 1, without subsequent treatment. FIG. 7B is a neurofilament stained (anti-NF200) tissue section of a rat sciatic nerve produced as described in Example 1 after treatment with a nerve autograft. FIG. 7C is a neurofilament stained (anti-NF200) tissue section of a rat sciatic nerve produced as described in Example 1 after treatment with an acellular porcine nerve. FIG. 7D is a neurofilament stained (anti-NF200) tissue section of a rat sciatic nerve produced as described in Example 1 after treatment with an acellular porcine artery. FIG. 7E is a neurofilament stained (anti-NF200) tissue section of a rat sciatic nerve produced as described in Example 1 after treatment with an acellular porcine artery filled with porcine acellular dermal matrix paste. FIG. 7F is a neurofilament stained (anti-NF200) tissue section of a rat sciatic nerve produced as described in Example 1 after treatment with a porcine acellular dermal matrix. Neurofilaments were demonstrated in sections treated with autografts, acellular porcine artery and acellular porcine nerve, but not in untreated controls and sections from animals treated with porcine acellular dermis.

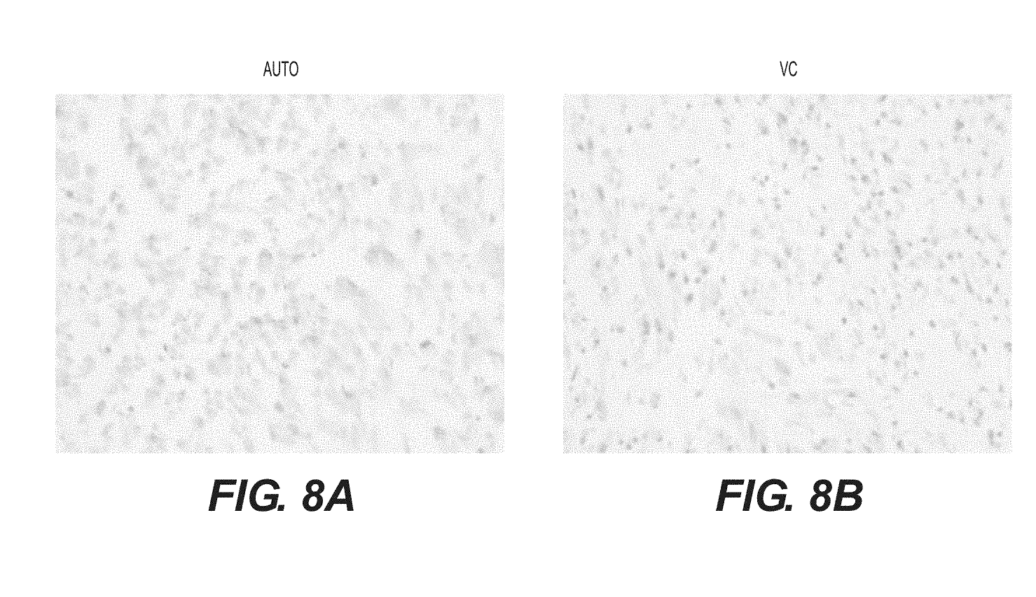

FIG. 8A is a tissue section of a rat sciatic nerve defect after treatment with a nerve autograft as described in Example 1 and stained with anti-GFAP (glial fibrillary acidic protein) antibodies that stain Schwann cells. FIG. 8B is a tissue section of a rat sciatic nerve defect after treatment with an acellular porcine artery as described in Example 1 and stained with stained with anti-GFAP antibodies. Many Schwann cells were identified in autografts sections and in sections from animals treated with acellular porcine artery.

Acellular porcine nerve and artery promoted nerve regeneration with histological features similar to those of autografts. In addition, acellular porcine artery provided overall better functional recovery based on recovery of gastrocnemius function (as assessed by dry weight of gastrocnemius) than any treated group other than autograft.

Initial experiments with acellular porcine artery seeded with stem cells demonstrated faster functional recovery than all above groups. By 4 months, all 5 animals implanted with artery and MSCs had gastrocnemius muscle weight 61-71% of the control's, indicating a good recovery of nerve function.

What is claimed is:

1. A method for treating a nerve, comprising:
selecting a recipient having a peripheral nerve with a defect across a portion of its length causing a loss of neural function; and
implanting an arterial tissue matrix across a region of the defect to produce a level of functional recovery of the lost neural function, wherein substantially all of the native cells have been removed from the arterial tissue matrix.

2. The method of claim 1, wherein the defect is greater than 1 cm in length.

3. The method of claim 1, wherein the defect is greater than 2 cm in length.

4. The method of claim 1, wherein the arterial tissue matrix is a porcine arterial tissue matrix.

5. The method of claim 4, including a treating step to remove α-1,3-galactose moieties from the arterial tissue matrix.

6. The method of claim 4, wherein porcine arterial tissue matrix is derived from a pig lacking expression of α-galactosyltransferase.

7. The method of claim 1, including a seeding step to colonize the arterial tissue matrix with an exogenous cell.

8. The method of claim 7, wherein the exogenous cell is autologous to the recipient.

9. The method of claim 7, wherein the exogenous cell is non-autologous to the recipient.

10. The method of claim 7, wherein the seeding step is performed before the implanting step.

11. The method of claim 7, wherein the seeding step is performed after the implanting step.

12. The method of claim 7, wherein the exogenous cell is a stem cell.

13. The method of claim 12, wherein the stem cell is a mesenchymal stem cell.

14. The method of claim 1, wherein the level of functional recovery is at least a 50% functional recovery of the lost neural function.

15. The method of claim 9, wherein the level of functional recovery is at least a 60% functional recovery of the lost neural function.

16. The method of claim 10, wherein the level of functional recovery is at least a 70% functional recovery of the lost neural function.

17. The method of claim 11, wherein the level of functional recovery is at least a 80% functional recovery of the lost neural function.

18. The method of claim 14, wherein functional recovery is quantified using a quantity of an innervated muscle taken from the list of quantities consisting of a size, a volume, a strength, a dry weight, or a pain stimulus response.

19. The method of claim 15, wherein functional recovery is quantified using a quantity of an innervated muscle taken from the list of quantities consisting of a size, a volume, a strength, a dry weight, or a pain stimulus response.

20. The method of claim 16, wherein functional recovery is quantified using a quantity of an innervated muscle taken from the list of quantities consisting of a size, a volume, a strength, a dry weight, or a pain stimulus response.

21. The method of claim 17, wherein functional recovery is quantified using a quantity of an innervated muscle taken from the list of quantities consisting of a size, a volume, a strength, a dry weight, or a pain stimulus response.
